(19) United States
(12) Patent Application Publication
(10) Pub. No.: US 2023/0248944 A1
(43) Pub. Date: Aug. 10, 2023

(54) Catheter Fixation Deivce

(71) Applicant: Board of Regents, The University of Texas System, Austin, TX (US)

(72) Inventor: JORGE LOPERA, San Antonio, TX (US)

(21) Appl. No.: 18/012,644

(22) PCT Filed: Jun. 24, 2021
(86) PCT No.: PCT/US21/38852

§ 371 Date: Dec. 22, 2022

Related U.S. Application Data
(60) Provisional application No. 63/043,170, filed on Jun. 24, 2020.

(51) Int. Cl. A61M 25/02; A61J 15/00

(52) U.S. Cl. CPC A61M 25/02; A61J 15/0053; A61M 2025/0286

(57) ABSTRACT

An external fixation system for percutaneous drainage procedures may include a base with a first and second side, a first coupler couplable to a conduit, and a second coupler couplable to a drainage bag or mechanism to flush or evacuate a drainage tube during use. The system may be modified to include a locking mechanism to secure conduit sutures. The system may also be used to convert a standard drainage tube into a low profile drainage tube. Also disclosed herein are methods and devices for converting standard feeding tubes into low profile feeding tubes.

### PRIORITY

This Application claims priority to U.S. Provisional Application Ser. No. 63/043,170 filed Jun. 24, 2020, which is incorporated herein by reference in its entirety.

### BACKGROUND

### 1. Field of the Invention

The invention generally relates to drainage tubes and specifically to methods of securing such tubes to patients.

### 2. Description of the Relevant Art

Drainage tubes (such as catheters) can be inserted into a body cavity, duct, or vessel, and are used to allow drainage and, in some instances allows the administration of fluids to the patient. In most uses, drainage tubes are thin, flexible tubes, though in some uses, they may be a larger, solid tube. A drainage tube is left inside the body, either temporarily or permanently, may be referred to as an indwelling drainage tube.

Many patients require long-term drainage procedures for diversion of the bile or urine. The current use of sutures and external fixation system to secure the drainage tube is not optimal due to the need for retention sutures with associated risks of pain and discomfort, frequent drainage tube dislodgement, skin infection and psychological reminder for the patient of the chronic disease by the presence of the external tube. Thus, there is a need to develop more convenient ways of implementing, securing, modifying, and replacing drainage tubes during long term treatments.

### SUMMARY

Certain aspects of the present disclosure relate to modifications to an external percutaneous fixation system for percutaneous drainage procedures. The unmodified external percutaneous fixation system comprises a base with couplers on each of two sides and a lumen of the base and couplers defined by the interior walls of the base and couplers.

The inventors discovered a solution to address issues with the current use of sutures and external fixation systems to secure the drainage tubes. The solution resides in modifications to the external percutaneous fixation system. In particular, modifications can include novel suture-locking components and mechanisms of the system and/or methods and devices for converting existing standard tubes for uses in medical procedures to low profile tubes for uses in medical procedures.

An external fixation system for percutaneous drainage procedures comprising: a base, comprising: a first side, wherein the first side is positioned adjacent an exterior surface of a subject during use; a first coupler coupled to the first side of the base; a second side opposite the first side; and a lumen defined by interior walls of the base and couplers such that the first coupler is in fluid communication with the second side of the base, wherein the first coupler is couplable to a conduit during use such that the conduit is in fluid communication with the second side of the base.

Other embodiments of the invention are discussed throughout this application. Any embodiment discussed with respect to one aspect of the invention applies to other aspects of the invention as well and vice versa. Each embodiment described herein is understood to be embodiments of the invention that are applicable to all aspects of the invention. It is contemplated that any embodiment discussed herein can be implemented with respect to any method or composition of the invention, and vice versa. Furthermore, compositions and kits of the invention can be used to achieve methods of the invention.

The use of the word “a” or “an” when used in conjunction with the term “comprising” in the claims and/or the specification may mean “one,” but it is also consistent with the meaning of “one or more,” “at least one,” and “one or more than one.”

Throughout this application, the term “about” is used to indicate that a value includes the standard deviation of error for the device or method being employed to determine the value.

The use of the term “or” in the claims is used to mean “and/or” unless explicitly indicated to refer to alternatives only or the alternatives are mutually exclusive, although the disclosure supports a definition that refers to only alternatives and “and/or.”

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

As used herein, the terms “comprises,” “comprising,” “includes,” “including,” “has,” “having,” “contains”, “containing,” “characterized by” or any other variation thereof, are intended to encompass a non-exclusive inclusion, subject to any limitation explicitly indicated otherwise, of the recited components. For example, a chemical composition and/or method that “comprises” a list of elements (e.g., components or features or steps) is not necessarily limited to only those elements (or components or features or steps), but may include other elements (or components or features or steps) not expressly listed or inherent to the chemical composition and/or method.

As used herein, the transitional phrases “consists of” and “consisting of” exclude any element, step, or component not specified. For example, “consists of” or “consisting of” used in a claim would limit the claim to the components, materials or steps specifically recited in the claim except for impurities ordinarily associated therewith (i.e., impurities within a given component). When the phrase “consists of” or “consisting of” appears in a clause of the body of a claim, rather than immediately following the preamble, the phrase “consists of” or “consisting of” limits only the elements (or components or steps) set forth in that clause; other elements (or components) are not excluded from the claim as a whole.

As used herein, the transitional phrases “consists essentially of” and “consisting essentially of” are used to define a chemical composition and/or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Various components may be described as “configured to” perform a task or tasks. In such contexts, “configured to” is a broad recitation generally meaning “having structure that” performs the task or tasks during operation. As such, the component can be configured to perform the task even when the component is not currently performing that task. Various components may be described as performing a task or tasks, for convenience in the description. Such descriptions should be interpreted as including the phrase “configured to.”

Unless defined otherwise, all technical and scientific terms used herein have the same meaning as commonly understood by one of ordinary skill in the art.

The term “connected” as used herein generally refers to pieces which may be joined or linked together.

The term “coupled” as used herein generally refers to pieces which may be used operatively with each other, or joined or linked together, with or without one or more intervening members.

The term “directly” as used herein generally refers to one structure in physical contact with another structure, or, when used in reference to a procedure, means that one process effects another process or structure without the involvement of an intermediate step or component.

Other objects, features and advantages of the present invention will become apparent from the following detailed description. It should be understood, however, that the detailed description and the specific examples, while indicating specific embodiments of the invention, are given by way of illustration only, since various changes and modifications within the spirit and scope of the invention will become apparent to those skilled in the art from this detailed description.

### DESCRIPTION OF THE DRAWINGS

The following drawings form part of the present specification and are included to further demonstrate certain aspects of the present invention. The invention may be better understood by reference to one or more of these drawings in combination with the detailed description of the specification embodiments presented herein.

FIGS. 1A-1B depict embodiments of an unmodified external fixation system for percutaneous drainage procedures.

FIGS. 2A-2B depicts a side view (FIG. 2A) and a perspective view (FIG. 2B) of an embodiment of an external fixation system for percutaneous drainage procedures, wherein the external fixation system is coupled to a drainage catheter.

FIGS. 3A-3B depicts an embodiment of an external fixation system for percutaneous drainage procedures, wherein the external fixation system is coupled to a drainage catheter using sutures, and wherein the sutures are unsecured (FIG. 3A), and an embodiment of an external fixation system for percutaneous drainage procedures, wherein the external fixation system is coupled to a drainage catheter using sutures, and wherein the sutures are secured (FIG. 3B).

FIG. 4 depicts an embodiment in which a standard drainage tube is converted by a modified external fixation system for percutaneous drainage procedures to a drainage tube having lower profile compared to the unconverted standard drainage tube.

FIG. 5 depicts a low profile conversion device.

FIG. 6 depicts the method in which the low profile conversion device is coupled to a feeding tube to produce a low profile feeding tube.

FIG. 7 depicts the dimension and perspective views of one example of an adaptor.

FIG. 8 depicts an adaptor with a valve, a metal connector pierces the valve to allow passage of the enteral feeding solutions.

FIG. 9 depicts a method of converting a PEG tube to a low profile tube using a torso model.

### DESCRIPTION

The following discussion is directed to various embodiments of the invention. The term “invention” is not intended to refer to any particular embodiment or otherwise limit the scope of the disclosure. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be an example of that embodiment, and not intended to intimate that the scope of the disclosure, including the claims, is limited to that embodiment.

### I. Unmodified External Fixation System

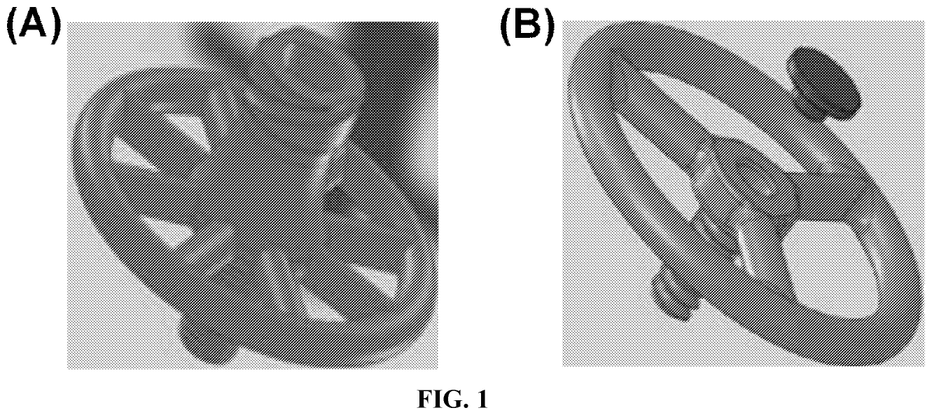

FIGS. 1A and 1B depict embodiments of external fixation system for percutaneous drainage procedures. External fixation system for percutaneous drainage procedures may include a base. The base may include first side. The first side may be positioned adjacent an exterior surface of a subject during use. The base may include first coupler coupled to first side of base. The base may include second side opposite the first side. The base may include a lumen defined by the interior walls of the base and couplers such that the first coupler is in fluid communication with the second side of the base. The first coupler may be couplable to conduit during use such that the conduit is in fluid communication with the second side of the base.

In some embodiments, portions of the external fixation system may be formed from metallic and/or non-metallic materials. An external fixation system may be made of various materials including, but are not limited to, metals, metal alloys, silicon, plastic, polymers, ceramics and combinations thereof. Some external fixation system embodiments may include components made of materials that can be autoclaved and/or chemically sterilized. Some components of an external fixation system may be formed of materials unable to be autoclaved and/or chemically sterilized. Components unable to be autoclaved and/or chemically sterilized may be made of sterile materials and placed in working relation to other sterile components during assembly of an external fixation system. In some embodiments, an entire external fixation system may be made of materials that can be autoclaved and/or chemically sterilized so that the external fixation system is a reusable instrument. In other external fixation system embodiments, all or selected components of the external fixation system may be made of sterile, disposable materials so that all or components of the external fixation system are designed for single use.

In some embodiments, the base includes a rim extending out and around at least a portion of the base. The rim may function as a type of flange. The rim may function to inhibit the base from entering an interior of the subject as the rim will be larger than the opening through the skin and/or body wall of the subject. The rim may be in any number of shapes. The shape depicted in the accompanying drawings of a rim is that of a substantially circular shape but this should not be seen as limiting.

In some embodiments, the system may include two or more radial arms extending from the base to the rim. The internal surfaces of the base, rim and radial arms may comprise the surfaces of two or more openings. The two or more openings may facilitate coupling of the base to the exterior surface of the subject during use. The two or more openings may allow fasteners to be used to couple the rim and thereby the base to a subject. For example, sutures may be looped through the openings coupling the rim to a subject. Other fasteners may be used, such as staples. In some embodiments, the base may be coupled to a subject using a hydrocolloid layer configured to absorb excess moisture around a perimeter of the base. In some embodiments, the base may be coupled to a subject using a double-sided hydrophilic adhesive layer configured to absorb excess moisture around the perimeter of the base. In some embodiments, the base may be coupled to a subject using a double-sided hydrophobic adhesive layer configured to repel excess moisture around the perimeter of the base.

The base may include two or more openings such that cleaning of the exterior surface of the subject is facilitated during use. The openings may allow cleaning of an entry site at the skin level. The openings may allow a practitioner easier access to the skin below the base and rim allowing the practitioner to more easily disinfect the tissue around the base and rim.

In some embodiments, the base may include a membrane or luer lock connection in the second side of the base to allow flushing of the tubes and/or connecting to an external drainage bag if needed. The base may include a fixation screw or lock in system to attach the base to the existing drain.

In some embodiments, the base may include a first coupler coupled to the first side of the base. In some embodiments, the first coupler is based upon a coupling mechanism including screw threads or coupling projections (e.g., teeth). Different attachments will allow the system to work with drains from 8-16 Fr. Once a conduit (e.g., a drainage catheter) is placed in the subject, an external portion is cut with sterile scissors. The cut end of the conduit may then be coupled to the base using the first coupler (e.g., a screw or a push in system). The base may be low profile and around 2 cm in diameter. The membrane in the center of the screw or fixation system will allow the patient to access the drain in case of blockage for flushing. In some embodiments, the base may be decoupled from the conduit. To exchange the conduits, the base is detached by, for example, either unscrewing or cutting the conduit adjacent to the base.

In some embodiments, the second side of the base may include a second coupler configured to facilitate the attachment of drainage bag or a mechanism to flush or evacuate a drainage catheter during use. In FIG. 1A the second coupler includes a luer lock type of connection on the second side of the base to allow flushings or connection to a drainage bag. In some embodiments, the second coupler may include a coupling mechanism including external screw threads, internal screw threads, a female luer fitting, a male luer fitting, a push-to-connect fitting, or a spring-tension loaded trap-door fitting.

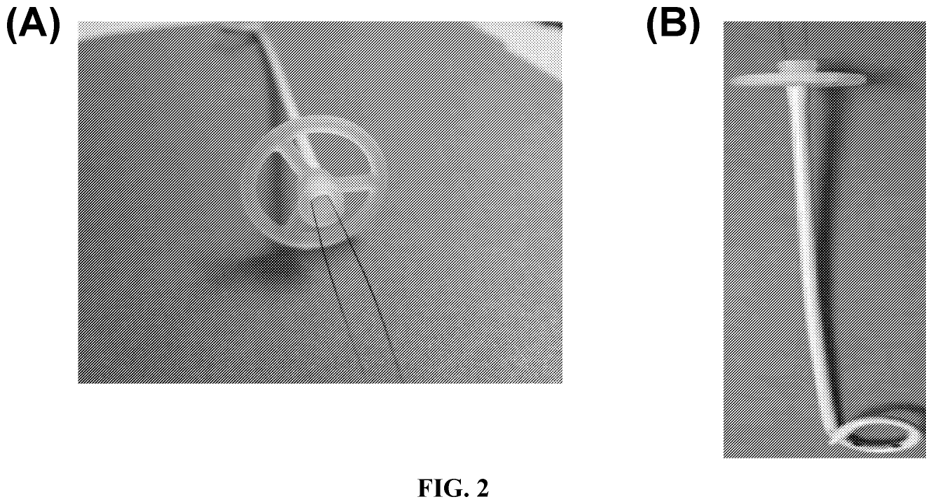

FIG. 2 depicts an embodiment of external fixation system for percutaneous drainage procedures, wherein the external fixation system is coupled to conduit. Specifically as depicted in these embodiments, the conduit includes a drainage catheter. Drainage catheters may function to drain excess and/or unwanted fluids out of a subject. Drainage catheters may allow care givers a means to evaluate a subject's health at least as it relates to the drainage site and the fluids drained. The drainage catheter may be at least semi-flexible so as not to harm a subject upon insertion. The drainage catheter may include a plurality of openings such that during use fluids may enter the catheter through the openings such that the fluids may be conveyed out of the subject through the catheter.

### II. Modified External Fixation System with Suture-Locking Mechanisms

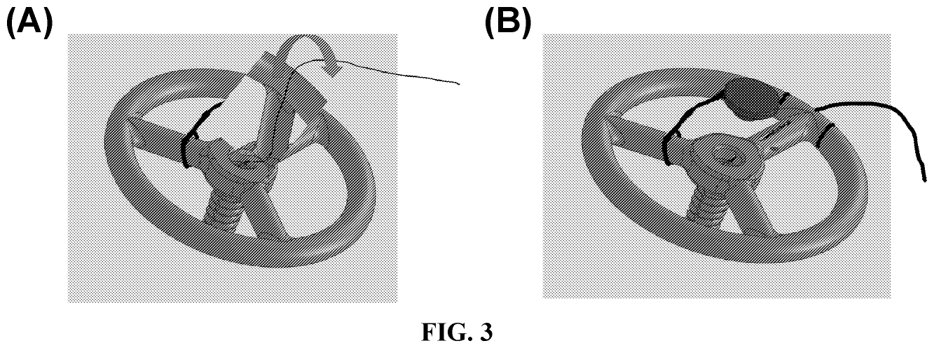

FIG. 3A depicts unsecured sutures extending through the conduit and from the lumen of the base. FIG. 3B depicts an embodiment of a modified external fixation system for percutaneous drainage procedures having suture-locking components and mechanisms. In some embodiments, the conduit comprises retention sutures used to secure a conduit to the base. One or more radial arms extending from the base to the rim comprise suture channels which are substantially perpendicular to the long axis of the lumen of the base. The one or more radial arms having suture channels can be hinged with respect to the base such that the arm is movable between an opened or closed state. Sutures extend through the conduit and base lumen and into a suture channel of a radial arm when the arm is lifted. Once the sutures are completely threaded through the suture channel and extend beyond the distal end of the arm, the arm is closed such that it is flush with the rim. As shown in FIG. 3B, closing the arm locks the sutures in place and prevents conduit dislodgment and internal migration.

In some embodiments, the sutures are secured to the system via a groove in one side of the system, which allows passage of the sutures to one side of the system. The sutures are then passed through a suture channel of a suture-securing device. The suture-securing device is coupled to a radial arm of the system. In some embodiments, the suture-securing device snaps on to an arm of the system which is configured to receive the suture-securing device. In some embodiments, the sutures are secured to the system knotting the sutures to a transverse bar in the second side of the system. In some embodiments, one suture is secured to the system, and a second suture exits the side of the system and is locked in place by pressing on a securing bar that holds the suture in place.

### III. Low Profile Drainage Tube Conversion

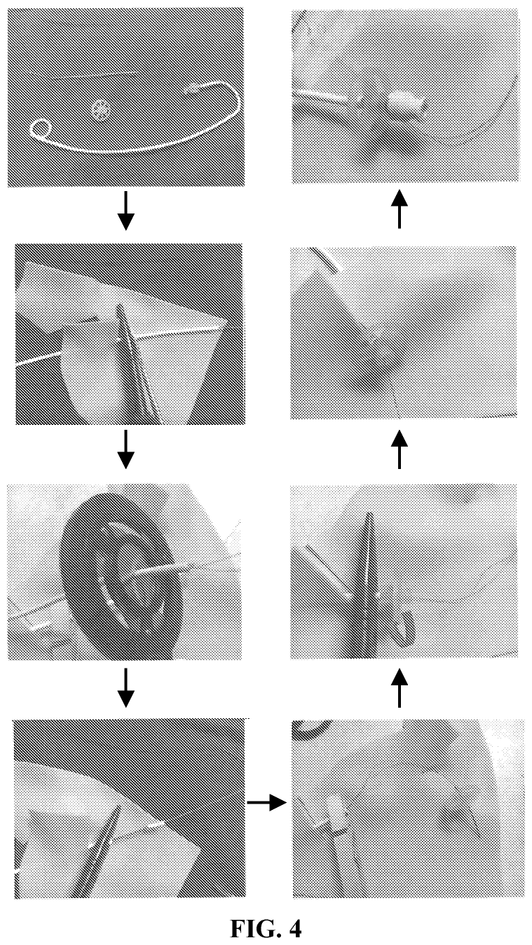

FIG. 4 depicts an embodiment in which a standard drainage tube is converted by an external fixation system for percutaneous drainage procedures to a drainage tube having a lower profile compared to the standard drainage tube. Specifically as depicted in this embodiment, the drainage tube can include but is not limited to a drainage catheter.

Disclosed herein are methods for converting a standard drainage tube using an external fixation system for percutaneous drainage procedures to produce a drainage tube that is lower in profile as compared to the unconverted standard drainage tube. In some embodiments, the system is configured to convert drainage tubes from 8-16 Fr. In some embodiments, the system is approximately 2 cm in diameter.

The standard drainage tube is gently retracted until it is positioned approximately 5 mm from the final desired position. The tube is clamped in place along the skin of the patient. In some embodiments, a special clamp having a slanted groove is used to hold the proximal portion of tube in place close to the skin of the patient and a larger lateral lumen to facilitate later coupling of the system. A stiff, protective (brace, collar, sheath, device) is inserted into the tube until the device is positioned just above where the tube is clamped. In some embodiments, the stiffener device is pre-inserted into the tube prior to practicing the disclosed methods. The retention sutures threaded through the drainage tube are surrounded by the stiffener device to prevent cutting the sutures when the drainage tube is cut. In some embodiments, the stiffener device can be made of plastic, metal, or a combination thereof. In some embodiments, the stiffener device is made of plastic in the distal portion and reinforced plastic and/or metal in the proximal portion.

The drainage tube is cut at a desired point between the proximal and distal end of the stiffener device. The tube is cut in a circular fashion. In some embodiments, scissors are used to cut the tube. In some embodiments, a special blade is used to cut the tube. The portion of the tube which has been cut away and stiffener device are removed, leaving in place the retention sutures threaded through the drainage tube.

The external fixation system for percutaneous drainage procedures is advanced over the sutures and coupled to the drainage tube. In some embodiments, the system is coupled to the drainage tube by the first coupler of the system using a push-to-connect fitting that locks the system in place. In some embodiments, the system is coupled to the drainage tube using a luer lock type of connection to allow connection to a drainage bag wherein the system is locked in place by rotating the system in a clockwise direction. In some embodiments, the coupling mechanism can include external screw threads, internal screw threads, a female luer fitting, a male luer fitting, or a spring-tension loaded trap-door fitting. The two sutures extend from the second side of the system and are secured using one or more of the methods described herein. The excess length of the sutures can be cut.

In some embodiments, a cap is attached to the second side of the system to allow the drainage tube to be flushed or to connect a drainage bag.

The system is coupled to the skin of the patient. In some embodiments, the system is sutured to the skin of the patient. In some embodiments, the system adhered to the skin of the patient.

To exchange the drainage tube, the system is detached by either unscrewing or cutting the drainage tube at the base of the system.

In some embodiments, the system is pre-attached to the drainage tube. The desired length of tube is selected based on the patient's preexisting drainage tube. In some embodiments, different lengths and diameters of tubes are utilized to meet patients' needs.

### IV. Conversion of Feeding Tube to C-Tube

Disclosed herein are devices and methods for converting standard feeding tubes to lower profile feeding tubes. Feeding tubes can include nasal tubes, gastric tubes (G-tubes) including percutaneous endoscopic gastrostomic (PEG) tubes, gastro-jejunal tubes, and jejunal tubes. PEG tubes refer to long, one-piece gastric tubes (G-tubes) placed by endoscopy. PEG tubes are often used as the initial G-tube for the first 8-12 weeks post-surgery. To place a PEG tube, a physician places an endoscope into the mouth. The endoscope is then advanced through the and into the stomach. The proximal end of the PEG tube rests in the stomach and is held in place either by a retention balloon or by an internal bumper. The distal end of the PEG tube comes out of the skin of the abdomen through a base connected to an external bumper, and a length of the tubing extends past the external bumper and terminates in an adapter for enteral feedings. Low profile G-tubes are preferred but are difficult and expensive to place de novo. Converting a PEG tube to a low profile G-tube can also be difficult, as the internal bumper holding the tube inside the patient's stomach is difficult to remove. Further, conversion requires a precise measurement of the distance of the tract, and a large inventory of tubes of different calibers and lengths is necessary as the tract distance will vary from patient to patient.

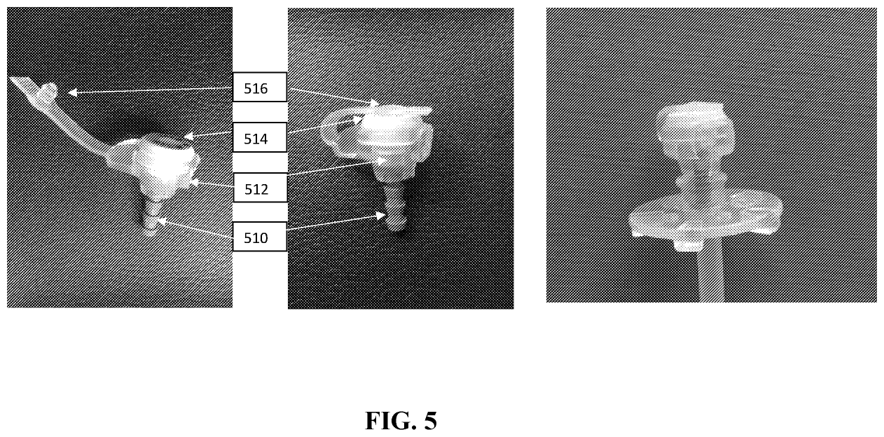

FIG. 5A depicts various views of a low profile conversion device. In some embodiments, the low profile conversion device can be used to convert a standard feeding tube to a low profile feeding tube. As shown in FIG. 5A, the device comprises a fitting 510 for insertion into a standard feeding tube. In some embodiments, the fitting can be tapered. In some embodiments, the fitting can comprise barbs to secure the fitting and prevent tube migration. In some embodiments, the fitting is available in different calibers for attachment to different size feeding tubes, for example, PEG tubes ranging from 12-24 Fr.

The fitting is connected to a base 512 comprising a coupler 514 configured to facilitate the attachment of a feeding pump. In some embodiments, the coupler includes a luer lock type of connection to allow connection to a feeding pump. In some embodiments, the coupler may include a coupling mechanism including external screw threads, internal screw threads, a female luer fitting, a male luer fitting, a push-to-connect fitting, or a spring-tension loaded trap-door fitting. In some embodiments, the coupler is locked such that the feeding tube of the feeding pump is locked into the system by turning and locking the coupler or by snapping the coupler into place. The device may include a lumen, the lumen defined by the interior walls of the coupler and fitting.

A closure mechanism 516 can be fitted over the coupler. In some embodiments, the closure mechanism includes a detachable soft plug to block the lumen of the coupler when the device is not in use. In some embodiments, the closure mechanism includes a hinged attachment which slides over the coupler and is configured to move between an open and closed state wherein when open, the coupler lumen is exposed, and when closed, the coupler lumen is covered.

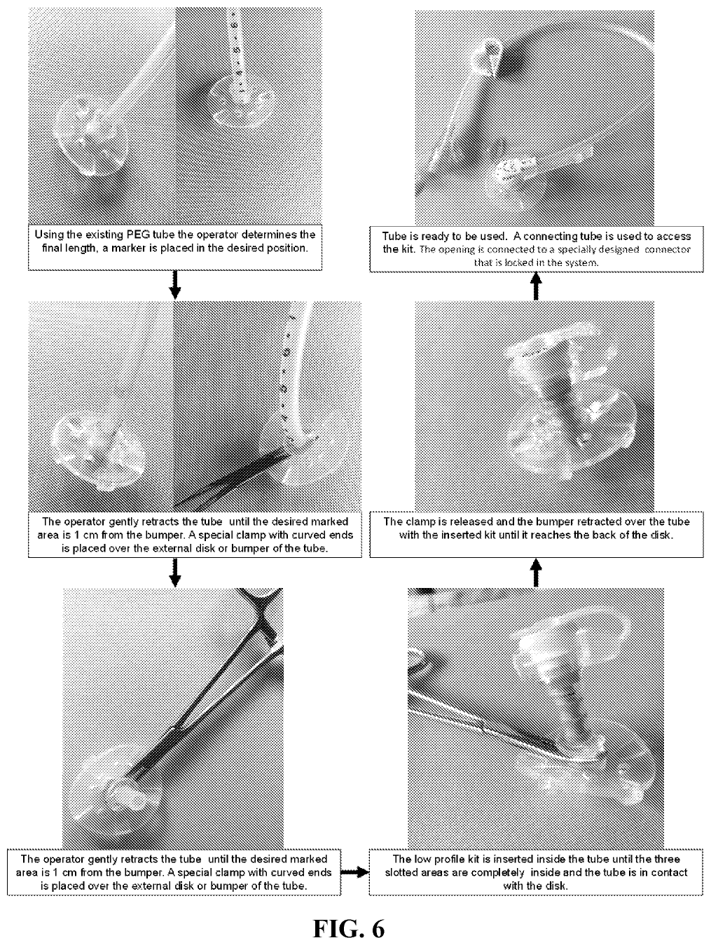

FIG. 6 depicts an embodiment in which a low profile conversion device is coupled to a feeding tube to produce a low profile feeding tube. The device allows a physician to convert any standard feeding tube, which has an excess of tubing extending beyond the exterior surface of a subject, into a low profile tube, providing extra comfort to the subject and saving significant health expenses.

Disclosed herein are methods for coupling a low profile conversion device to an standard feeding tube to produce a low profile feeding tube. In some embodiments, the standard feeding tube comprises an internal bumper resting within the stomach, an external base connected to an external bumper attached to the skin of the abdomen, and a tube extending from the internal bumper through the wall of the stomach and wall of the abdomen, through the base and external bumper, and beyond the skin of the abdomen. A desired final length of the feeding tube from the proximal end of the tube resting in the stomach to the distal end extending beyond the skin of the abdomen is determined. A marker is placed in the desired position on the portion of the feeding tube extending beyond the skin of the abdomen corresponding to the desired final length of the tube. In some embodiments, the standard feeding tube has preexisting markings which can be used to indicate the desired position corresponding to the desired length of the tube.

After the feeding tube is marked to indicate a desired length, the feeding tube is gently retracted from within the body until the marked area is approximately 1 cm from the bumper. The standard feeding tube is clamped between the external base and the bumper. In some embodiments, a special clamp with curved ends is used to clamp the standard tube between the base and the bumper. After being clamped, the feeding tube is cut at the marked area, and conversion device disclosed herein is coupled to the tube to produce a low profile feeding tube. In some embodiments, the conversion device is inserted into the feeding tube until the fitting is completely sheathed by the tube and in contact with the base of the conversion device. After the conversion device is coupled to the cut tube, the clamp is released from between the base and bumper of the tube, and the external bumper of the tube is retracted over the tube containing the coupled device until the external bumper is flush against the base of the conversion device.

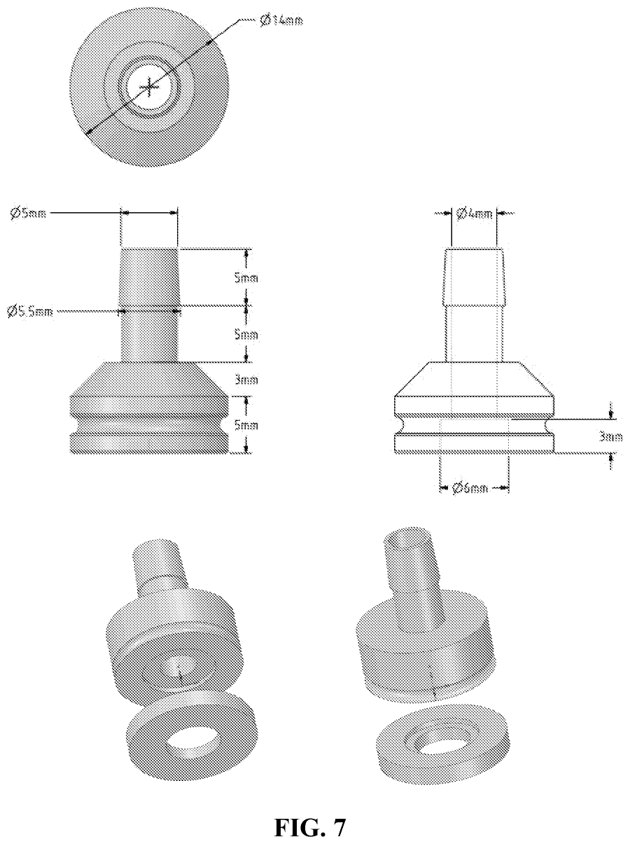

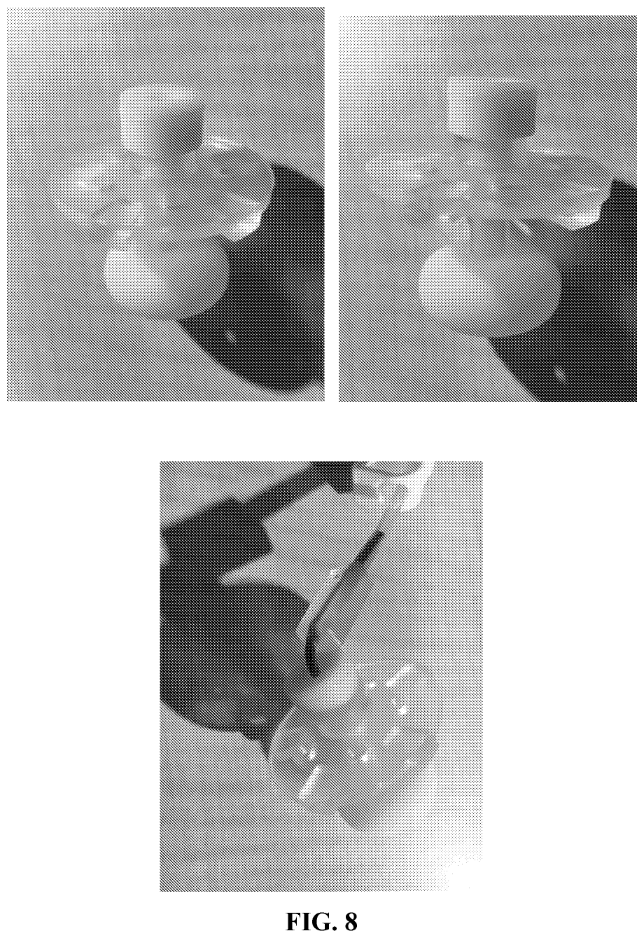

FIG. 7 depicts one example of an adapter and provides dimensions for one embodiment of the adapter. FIG. 8. Depicts an adapter having a tube connecting a bumper to a securement device having an adapter connected to the securement device. The adaptor has a valve, a connector pierces the valve to allow passage of the enteral feeding solutions.

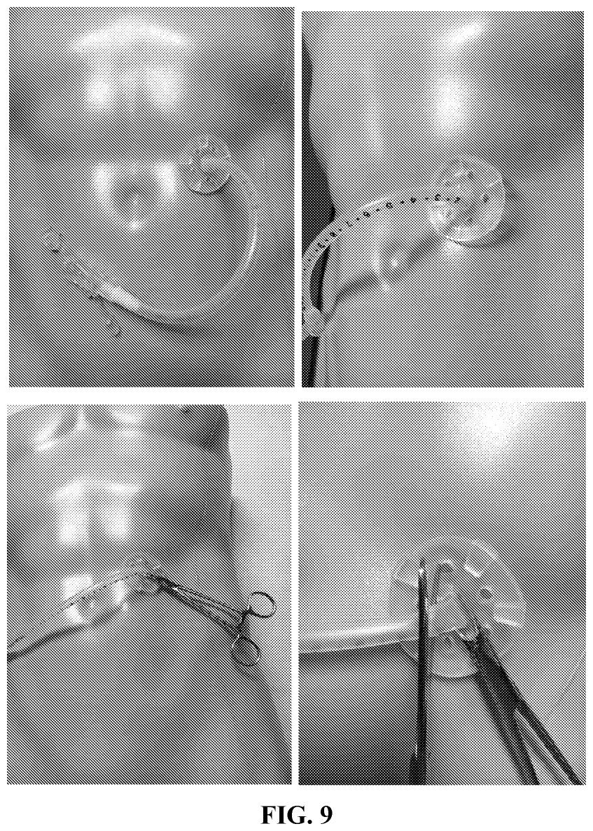

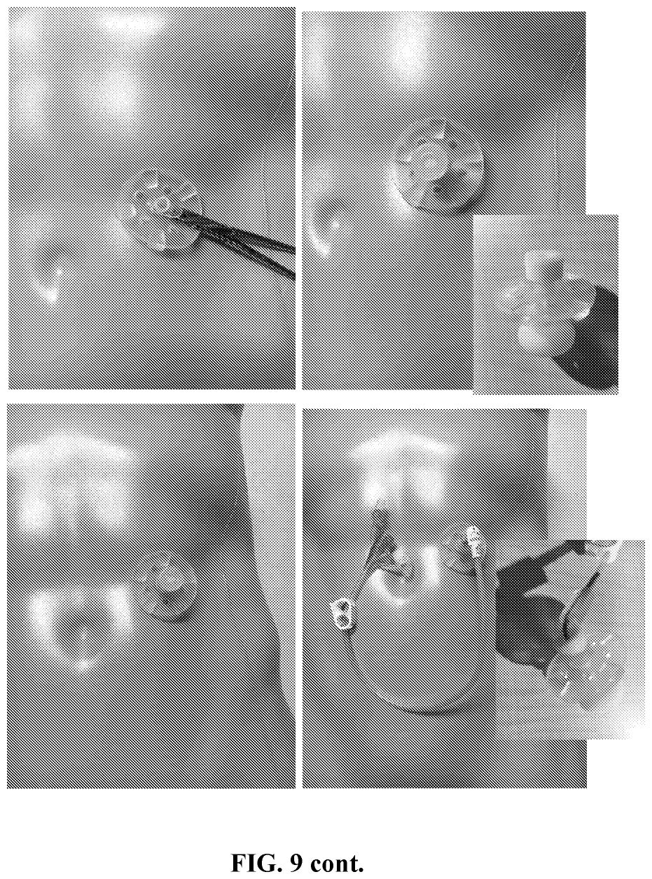

FIG. 9 shows the method of installing an adaptor using a torso model having an existing PEG tube. A clamp is placed and the existing tube is cut. Adapter is placed inside the tube and an internal bumper is moved over the adapter. To finalize installation a low profile feeding tube is inserted through the valve of the adapter, wherein the terminus of the feeding tube pierces the valve and allows the passage of enteral feeding solutions.

1. An external fixation system for percutaneous drainage procedures comprising:
a base, comprising:
a first side, wherein the first side is positioned adjacent an exterior surface of a subject during use;
a first coupler coupled to the first side of the base;
a second side opposite the first side; and
a lumen defined by interior walls of the base and couplers such that the first coupler is in fluid communication with the second side of the base,
wherein the first coupler is couplable to a conduit during use such that the conduit is in fluid communication with the second side of the base.

2. The system of claim 1, wherein the base comprises a metallic and/or a non-metallic material.

3. The system of claim 1 or 2, wherein at least a portion of the conduit extends into an interior of a subject.

4. The system of claims 1-3, wherein the conduit comprises a drain tube.

5. The system of claims 1-3, wherein the conduit comprises a catheter.

6. The system of claims 1-5, wherein the base further comprises a rim extending out and around at least a portion of the base.

7. The system of claim 6, wherein the rim is configured to inhibit the base from entering an interior of the subject.

8. The system of claim 6 or 7, wherein the rim comprises one or more openings such that the base is coupled to the exterior surface of the subject.

9. The system of claims 6-8, wherein the rim comprises one or more openings such that cleaning of the exterior surface of the subject is facilitated during use.

10. The system of claims 6-9, wherein one or more radial arms extending from the rim to the base.

11. The system of claim 10, wherein one or more of the radial arms are hinged with respect to the base such that the arm is movable between an open and closed state.

12. The system of claim 10 or 11, wherein when a radial arm is in a closed state, the radial arm is flush with the rim.

13. The system of claims 10-12, wherein one or more of the radial arms comprise suture channels.

14. The system of claims 1-13, wherein the conduit comprises retention sutures to secure the conduit to the base.

15. The system of claims 13, wherein the suture channels are substantially perpendicular to the long axis of the lumen of the base.

16. The system of claims 13-15, wherein the suture channels are configured to receive retention sutures extending from the conduit and through the lumen when a radial arm comprising a suture channel is in an open state.

17. The system of claims 13-16, wherein the retention sutures are secured to the base when a radial arm comprising a suture channel through which retention sutures extend is in a closed state.

18. The system of claims 1-17, wherein the first coupler is fluidly connected to the conduit via screw threads.

19. The system of claims 1-17, wherein the first coupler is fluidly connected to the conduit via teeth.

20. The system of claims 1-19, wherein the second side of the base comprises a second coupler configured to facilitate the attachment of a drainage bag or a mechanism to flush or evacuate a drainage catheter.

21. The system of claim 20, wherein the second coupler comprises external screw threads, internal screw threads, a female luer fitting, a male luer fitting, a push-to-connect fitting, or a spring-tension loaded trap-door fitting.

22. The system of claims 1-21, wherein the base is coupled to the exterior surface of the subject using sutures.

23. The system of claims 1-21, wherein the base coupled to the exterior surface of the subject using a hydrocolloid layer configured to absorb excess moisture around a perimeter of the base.

24. The system of claims 1-21, wherein the base is coupled to the exterior surface of the subject using a double-sided hydrophilic adhesive layer configured to absorb excess moisture around the perimeter of the base.

25. The system of claims 1-21, wherein the base coupled to the exterior surface of the subject using a double-sided hydrophobic adhesive layer configured to repel excess moisture around the perimeter of the base.

26. A method for converting a standard drainage tube in a subject using the external fixation system of claim 1, comprising:
retracting the standard drainage tube until it is positioned approximately 5 mm from a final desired position;
clamping the drainage tube in place along the exterior surface of the subject;
inserting a stiffener device into the tube until the device is positioned just above where the drainage tube is clamped, wherein the stiffener device prevents cutting of retention sutures within the drainage tube;
cutting the drainage tube at a desired point between the proximal and distal end of the stiffener device;
removing the portion of the drainage tube which has been cut away;
removing the stiffener device to expose the retention sutures within the drainage tube;
advancing the system over the exposed retention sutures;
coupling the external fixation system to the drainage tube by the first coupler of the system; and
securing the retention sutures to the base of the system.

27. The method of claim 26, wherein the standard drainage tube comprises drainage tubes from 8-16 Fr.

28. The method of claim 26 or 27, wherein the converted standard drainage tube is lower in profile as compared to the unconverted standard drainage tube.

29. The method of claims 26-28, wherein the clamp used for clamping the drainage tube along the exterior surface of the subject comprises a slanted groove and a lateral lumen at least 10% larger than the circumference of the first coupler of the system.

30. The method of claims 26-29, wherein the stiffener device is pre-inserted into the drainage tube prior practicing the method.

31. The method of claims 26-30, wherein the stiffener device is made of plastic, metal, or a combination thereof

32. The method of claims 26-31, wherein the stiffener device is made of plastic in a distal portion and reinforced plastic and/or metal in a proximal portion.

33. The method of claims 26-32, wherein the system is coupled to the drainage tube using a push-to-connect fitting, a luer lock type of connection, external screw threads, internal screw threads, a female luer fitting, a male luer fitting, or a spring-tension loaded trap-door fitting.

34. The method of claims 26-33, the method further comprising attaching a cap to the second side of the system.

35. The method of claims 26-34 further comprising coupling the drainage tube to the exterior surface of the subject after securing the retention sutures to the base of the system.

36. A conversion device to convert a standard feeding tube into a feeding tube having a lower profile as compared to the standard feeding tube comprising:
a base comprising a coupler, wherein the coupler is configured to facilitate the attachment of a feeding pump;
a fitting for insertion into a standard feeding tube coupled to the base; and
a lumen defined by interior walls of the base and fitting.

37. The device of claim 36, wherein feeding tubes ranging from 12-24 Fr are converted.

38. The device of claim 36 or 37, wherein the fitting is tapered.

39. The device of claim 36 or 37, wherein the fitting is barbed.

40. The device of claims 36-39, wherein the coupler comprises a push-to-connect fitting, a luer lock, external screw threads, internal screw threads, a female luer fitting, a male luer fitting, a push-to-connect fitting, or a spring-tension loaded trap-door fitting.

41. The device of claims 36-40, wherein the device comprises a closure mechanism.

42. The device of claim 41, wherein the closure mechanism comprises a detachable soft plug to block the lumen of the coupler.

43. The device of claim 41, wherein the closure mechanism comprises a hinged attachment configured to slide over the coupler and to move between an open and closed state, wherein when open, the coupler lumen is exposed, and when closed, the coupler lumen is covered.

44. A method for converting a standard feeding tube in a subject to a feeding tube having a lower profile using the conversion device of claim 36 comprising:
determining a desired final length of the feeding tube from a proximal end of the tube to a distal end of the tube;
placing a marker on the feeding tube to indicate a final desired length of the feeding tube at a marked area;
retracting the feeding tube from within the subject until the marked area is approximately 1 cm from an external bumper of the feeding tube on the exterior surface of the subject;
clamping the feeding tube between the external bumper of the feeding tube and an external base of the feeding tube;
cutting the feeding tube at the marked area;
coupling the conversion device to the feeding tube;
releasing the clamp from between the base and bumper of the feeding tube; and
retracting the bumper of the feeding tube over the coupled device until the bumper is flush against the base of the device.

45. The method of claim 44, wherein the standard feeding tube has preexisting markings to indicate a final desired length of the feeding tube at a marked area.

46. The method of claim 44 or 45, wherein the conversion device is coupled to the feeding tube by a push-to-connect mechanism comprising insertion of the device into the feeding tube.

47. The method of claim 46, wherein the conversion device is inserted into the feeding tube until the fitting is completely sheathed by the tube.
